(19) United States
(12) Patent Application Publication
(10) Pub. No.: US 2025/0000560 A1
(43) Pub. Date: Jan. 2, 2025

(54) THERMAL LITHOTRIPTER

(71) Applicant: GYRUS ACMI, INC D/B/A OLYMPUS SURGICAL TECHNOLOGIES AMERICA, Westborough, MA (US)

(72) Inventors: Aseem Singh, Northborough, MA (US); Kamaldeep Singh, Marlborough, MA (US)

(21) Appl. No.: 18/734,199

(22) Filed: Jun. 5, 2024

Related U.S. Application Data
(60) Provisional application No. 63/510,735, filed on Jun. 28, 2023.

(51) Int. Cl. A61B 18/00; G16H 40/63

(52) U.S. Cl. CPC A61B 18/00; G16H 40/63; A61B 2018/00095; A61B 2018/00577; A61B 2018/00642; A61B 2018/00714; A61B 2018/0072; A61B 2018/00732; A61B 2018/00767

(57) ABSTRACT

A lithotripsy device for use in a thermal lithotripsy procedure may include an elongate member, having a proximal end and a distal end and a first thermal lithotripsy transducer (e.g., a thermoelectric transducer such as a thermocouple) at the distal end of the elongate member. The first thermal lithotripsy transducer configured to heat and to cool, in response to an applied first electrical input signal, to emit a thermal lithotripsy dose toward a lithotripsy target.

### PRIORITY CLAIM

This application claims the benefit of priority to U.S. Provisional Patent Application Ser. No. 63/510,735, filed Jun. 28, 2023, the contents of which are hereby incorporated by reference in their entirety.

### TECHNICAL FIELD

The present disclosure relates to treatment of a calculus such as a kidney stone during a lithotripsy procedure. Specifically, the present disclosure relates to a lithotripsy device using a thermoelectric transducer to emit a thermal lithotripsy excitation toward a lithotripsy target.

### BACKGROUND

A Lithotripsy procedure can involve treatment of a lithotripsy target such as a calculus (e.g., a kidney stone or a gall stone), or treatment of a portion of tissue, such as for cutting or cauterizing tissue. Stones can be formed from different materials and may have different densities or levels of hardness or softness. For example, harder kidney stones can be formed from Calcium Oxalate or Calcium Phosphate, and softer kidney stones can be formed from Uric Acid. A kidney stone can have a softer outer “shell” and a harder core, or vice versa.

In such lithotripsy procedures, laser or electromagnetic energy or ultrasonic energy) can be emitted from a source such as a laser or an ultrasonic transducer to reduce stones into small fragments or to a dust-like consistency that can either be passed from the body naturally or actively removed using a retrieval device or by flushing using a solution such as saline.

### SUMMARY

This document describes systems and methods for stone ablation (lithotripsy) using thermal cycling (thermal lithotripsy). Thermal cycling (e.g., with a cycling time of five seconds or less) can cause harder stones, such as stones formed from Calcium Oxalate or Calcium Phosphate to break apart or to become dislodged from a portion of tissue using cycles of one or both of thermal expansion or contraction. The cycling time of the thermal cycle can be any time or duration desired or appropriate for the particular lithotripsy procedure. A thermal lithotripsy device for use in a thermal lithotripsy procedure can include an elongate or elongated member such as a catheter or a flexible portion of a scope (e.g., an endoscope) having a proximal end and a distal end. The lithotripsy device can be included as a part of or a component of an endoscopic system, which can include or be connected to an irrigation system, control circuitry or a similar controller or computer components, various sensors (e.g., temperature or pressure sensors), or the like. The scope can include a working channel, and the elongate member can be sized and shaped to be inserted via the working channel of the scope so as to emit a thermal lithotripsy dose toward the lithotripsy target from a location that is at or near the distal end of the scope.

The lithotripsy device can include one or more thermal lithotripsy transducers (e.g., one or more thermoelectric transducers, such as a thermocouple), at the distal end of the elongate member. A thermoelectric transducer can be configured to heat, to cool, or to both heat and to cool, in response to an applied electrical input signal. In an example, the transducer can be configured to actively heat and actively cool in response to the input signal. In another example, the transducer can be configured to actively heat and passively cool. Such thermoelectric heating and cooling can be used to emit a thermal lithotripsy dose toward a lithotripsy target, where such a thermal lithotripsy dose can include a cyclic heating and cooling capable of fragmenting the lithotripsy target. Thus, thermal lithotripsy can include heating and/or cooling (e.g., actively heating and/or actively cooling) the thermal lithotripsy transducer to produce a thermal lithotripsy dose at the thermal lithotripsy transducer. The thermal lithotripsy dose can then be emitted from the thermal lithotripsy transducer (e.g., as a thermal shockwave) toward the lithotripsy target to dislodge and/or ablate or break apart the target.

The thermoelectric transducer can include a first electrically conductive material, a second electrically conductive material, and a junction between the first electrically conductive material and the second electrically conductive material. The lithotripsy device can include or be connected to controller circuitry. The controller circuitry can be operable to control a pulsing of the electrical input signal at a particular frequency to operate the thermoelectric transducer for thermal lithotripsy of the lithotripsy target. The controller circuitry can control the electrical input signal to cause a thermal cycle to occur or recur, such as between a relatively higher temperature and a relatively lower temperature, for performing thermal lithotripsy of the lithotripsy target. For example, the thermal cycle can be between a first temperature of 0 degrees Celsius (° C.) and a second temperature of 50° C., or between other first and second temperatures within that range, inclusive.

The distal tip of the elongate member carrying the thermal lithotripsy transducer can be flat, convex, dome-shaped, semi-spherical, or the like, such as to accommodate multiple thermoelectric transducers. When multiple thermoelectric transducers are included at the distal tip or distal end of the scope, the individual ones of the multiple thermoelectric transducer can be operable independently of each other. For example, when a second thermoelectric transducer is included in the distal tip or end of the scope, the second thermoelectric transducer can be controlled by an applied second electrical input signal such that the second thermoelectric transducer is operable separately from or independently of the first thermoelectric transducer. Using thermoelectric transducers that are capable of operating independently of each other can allow for more precise heating and cooling. This, in turn, can permit a more targeted thermal lithotripsy dose being delivered to the target.

The amount of energy, such as a current or a voltage of the electrical input signal of a particular thermoelectric transducer can be established or adjusted, such as can include using duty cycle control and/or pulse width control of the electrical input signal to operate the thermoelectric transducer. The electrical or other input signals can include a square wave, a triangular wave, or any type of signal waveform desired or appropriate. The duty cycle or pulse width can be selected or controlled and can be used to control the thermal cycling of the thermoelectric transducer (e.g., a square wave with a 50% duty cycle). The pulse width and/or the duty cycle can be established or adjusted during the procedure, either automatically by an artificial intelligence (AI) or machine learning (ML) or other algorithm (e.g., a non-AI or non-ML deterministic algorithm) or process or manually by a physician. In an example, a process can operate semi-automatically, such as under human supervision. For example, the process can output a suggestion to the physician, which the physician can accept or reject as the physician desires. Additionally, or alternatively, the adjustment can be made using a hardware-based feedback loop.

A temperature sensor can be included. The temperature sensor can be configured to monitor a temperature at or near the distal end of the elongate member. The temperature sensor can further be configured to transmit a signal representing the monitored temperature to the control circuitry. Based on the monitored temperature, the control circuitry can adjust one or more electrical input signals to control one or more thermoelectric transducers, such as to heat and cool to emit the thermal lithotripsy dose toward the lithotripsy target. Additionally or alternatively, the process can receive or determine a thermal lithotripsy ablation parameter of the lithotripsy target. Based on the thermal lithotripsy ablation parameter, the control circuitry can adjust a frequency or period of the electrical input signal to the thermoelectric transducer to control the thermal cycle. The ablation parameter can include a size of the lithotripsy target, a composition of the lithotripsy target, a location of the lithotripsy target in the anatomy of a subject or patient, or the like. One or more ablation parameters of the lithotripsy target can be determined using spectral analysis of a response signal from the target, such as light returned back from the lithotripsy target in response to an illumination signal provided to the target. Examples of determining target characteristics are described in U.S. patent application Ser. No. 17/938,826, the contents of which are fully incorporated by reference into this application.

Advantages of a thermal lithotripsy device such as described herein can include providing another avenue or method for the treatment of stones. Because the heating and cooling using the thermoelectric transducer can be controlled using an electrical input signal, the cycle time for the thermoelectric transducer to alternate between hot and cold states and/or to dissipate the heat can be very fast. Thus, a temperature gradient of a thermal lithotripsy device can be easier to control than the temperature gradient of a lithotripsy device that uses another type of energy source, such as a laser. Similarly, the locus and effect of the thermal energy from a thermal lithotripsy device can be controlled more easily than energy from a laser lithotripsy device, since laser energy can be reflected off of certain surfaces, such as a reflective surface of another medical device being used, and by so doing, such laser energy can be delivered to unintended tissue.

Furthermore, using ultrasonic or laser energy in lithotripsy can cause a stone to break apart to an extent that the field of view can be so obscured that a physician may be required to spend extra time in the procedure to clear debris from the fragmented stones, which can add time to the overall procedure. Using a thermal lithotripsy dose may result in stones being more controllably fragmented, thus keeping the field of view clearer for the physician, which can help reduce overall procedure time. Finally, the disclosed lithotripsy device using one or more thermoelectric transducers in the tip of a scope may be smaller than lithotripsy devices that utilize other energy sources, which can help improve patient comfort during the procedure and can also help improve recovery after the procedure.

Throughout the present disclosure the terms thermal lithotripsy transducer and thermoelectric transducer may be used interchangeably. Similarly, the terms electrical input signal, applied electrical input signal, electrical signal, applied electrical signal, and input signal may be used interchangeably and may be understood to include an electric current or a voltage signal.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a distal end of an elongate member near a lithotripsy target and an example of a thermoelectric transducer.

FIG. 2 illustrates an example of a graph of a thermal cycle showing voltage and temperature changes of a thermoelectric transducer.

FIG. 3 illustrates an example of a method of ablating a lithotripsy target.

FIG. 4 includes an example of a flow diagram for ablating a lithotripsy target.

FIG. 5 is a block diagram illustrating an example of a machine upon which one or more embodiments may be implemented.

FIG. 6 illustrates a schematic diagram of an exemplary computer-based clinical decision support system (CDSS).

### DETAILED DESCRIPTION

A lithotripter is a device used to reduce or break apart targets such as kidney stones and ureteral stones. Lithotripters can be used to ablate (break up, break apart, etc.) targets using minimally-invasive surgical intervention. Lithotripters can mechanically ablate targets or can use energy or signals from an energy source, such as a laser or source of radiation energy, or a source of ultrasonic energy. Targets can vary in size, can be located in different portions of the body of a patient, and/or can be made or formed from different types of material and can thus have different levels of hardness or softness.

The thermal lithotripter disclosed herein can perform ablation of a target (e.g., a kidney stone) using thermal cycling of a thermoelectric transducer, such as a thermocouple, to deliver a thermal lithotripsy dose to the target. A slow thermal cycling can cause a target formed from a hard material such as Calcium Oxalate to break or become dislodged when the stone is located in a difficult location or portion of anatomy during cycles of thermal expansion and contraction (heating and cooling) using the thermocouple.

When an electrical input signal, such as an electric current, is applied to an input of the transducer, heat can be generated at one junction of the transducer and absorbed at another junction of the transducer. In another example, the electrical input signal can be a voltage input to the transducer or junction. The heating and cooling at an electrified junction of two different material conductors can be referred to as the Peltier effect. When a current is made to flow through a junction between two electrical conductors, heat can be generated or removed at the junction, which can cause a thermal cycle as the current is applied or terminated.

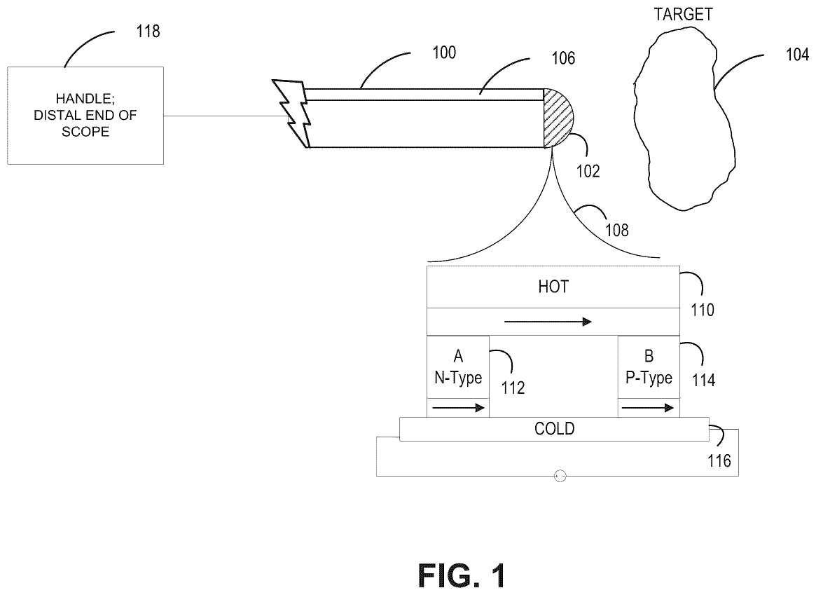

FIG. 1 illustrates an example of a distal end 100 of an elongate member near a lithotripsy target and an example of a thermoelectric transducer. In FIG. 1, a distal end 100 of an elongate member can include a tip 102 containing a thermoelectric transducer 108. The distal end 100 of the elongate member can be included as a part of a lithotripsy device included in or configured to be used with a scope, such as an endoscope, and used to emit a thermal lithotripsy dose toward lithotripsy target 104. For example, the elongate member can be included in or insertable via a flexible portion of an endoscope and can be connected to a handle at a proximal end of the elongate member. The elongate member can also include a channel 106. The channel 106 can span the length of the elongate member from a proximal end to the distal end 100. The proximal end of the scope can be connected to a handle or other components as illustrated by the box 118 at the left side of FIG. 1 (representing the proximal end of the scope and all components that may be connected to the scope). The channel 106 can include wiring. The wiring can be used to connect the thermoelectric transducer 108 to one or more other components such as a power source or signal generator, a controller or control circuitry, or the like. The lithotripsy target 104 can include a calculus, such as a kidney stone to be dislodged, reduced, and/or ablated during a lithotripsy procedure. The tip 102 of the elongate member can have a flat shape or can be convex, domed or semi-spherical shaped. The shape of the tip 102 of the elongate member can be based at least in part on the number of thermoelectric transducers included in the tip 102.

The thermoelectric transducer 108 can include a first junction 110 and a second junction 116. In an example, the first junction 110 can generate heat at a first end of the thermoelectric transducer 108 in response to an electrical input signal (e.g., an electric current) or a cycling of the electrical input signal. The second junction 116 can absorb heat at a second end of the thermoelectric transducer 108 in response to the electrical input signal (or a cycling of the electrical input signal). The first junction 110 and the second junction 116 can be arranged such that the first junction 110 is substantially opposite or across from the second junction 116. Between each junction, the thermoelectric transducer 108 can include a first conductive material 112 and a second conductive material 114 of different material types. For example, the first conductive material 112 can be an N-type conductor or semiconductor (e.g., Bismuth) and the second conductive material 114 can be a P-type conductor or semiconductor (e.g., Telluride). Bismuth and Telluride are examples of N and P type conductors that can be used in the thermoelectric transducer 108, however any P-type and N-type conductors capable of generating a Peltier effect can be used in the thermoelectric transducer 108. Thus, there can be net heating at the first junction 110 and heat can be absorbed at the second junction 116 and by the lithotripsy target 104. This heating and cooling results in thermal cycling or a thermal shock wave to break the lithotripsy target 104.

When an electrical input signal such as an electric current is passed through the circuit of the thermoelectric transducer 108 (as denoted by the arrows), heat can be generated at the first junction 110 and absorbed at the second junction 116. This is referred to as the Peltier effect, or the presence of heating or cooling at an electrified junction of two different electrical conductors. The Peltier heat generated at the junction per unit time is given by:

$$Q = ( Π A - Π B ) I \qquad Equation ( 1 )$$

In Equation 1, ΠA and ΠB are the Peltier coefficients of electrical conductors A and B (the first conductive material 112 and the second conductive material 114), and I is the electric current from A to B. The total heat generated by the thermoelectric transducer 108 can include Joule heating and thermal gradient effects, however, these contributions may be negligible or minimal compared with the Peltier heating and cooling.

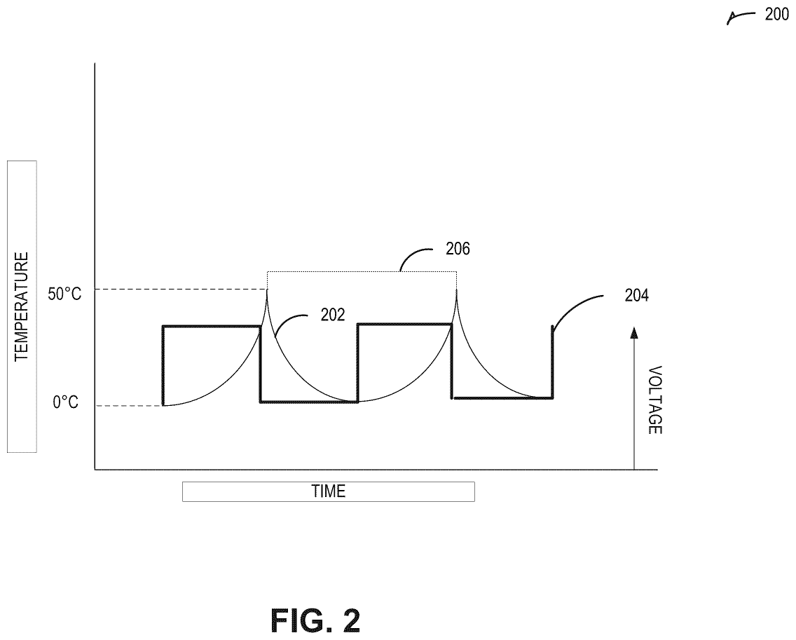

FIG. 2 illustrates an example of a temperature and voltage vs. time graph 200 of a thermal cycle showing voltage and temperature changes of a thermoelectric transducer. As illustrated in the graph 200, the Peltier junction temperatures (as shown on the temperature versus time trace 202) can cycle between 0° C. and 50° C., inclusive, as an electrical input signal such as a square wave is applied to the thermoelectric transducer 108. In FIG. 2, a square wave electrical input signal 204 can be applied to cause the temperature at the junction to rise from 0° C. to 50° C. and then lower from 50° C. to 0° C. as the current applied to the thermoelectric transducer turns ON and OFF.

The period of the thermal cycle 206 can be selected based on the amount of heating or cooling that is desired during the procedure (e.g., can be less than a second or a fraction of a second) and can be adjusted at any time or throughout the procedure as conditions change. For example, the input signal can be adjusted (e.g., by controller circuitry included in or coupled to the lithotripsy device) using duty cycle or pulse width control of the electrical input signal. Additionally, or alternatively, the amplitude of the input signal can be adjusted independent of or along with adjustment of the pulse width and/or the duty cycle. In an example, the input signal can be an input current. The value of the input current can be less than one Ampere (e.g., one-quarter Ampere), and the voltage of the input signal can move or oscillate between an upper and lower voltage value. For example, the voltage of the input signal can oscillate between five volts and 12 volts, inclusive.

The value of the current and/or the voltage can be selected and/or adjusted as desired or necessary or as appropriate based on conditions that arise during a medical procedure. For example, the input signal can be adjusted based, at least in part, on an ablation parameter of the lithotripsy target. The ablation parameter can include the size of the lithotripsy target, a location of the lithotripsy target, a material composition of the lithotripsy target, or the like. In an example, the lithotripsy target can be monitored at different times during the procedure to determine the ablation parameter(s) at a first time and a later second time, to determine whether a change in the lithotripsy target (e.g., a change in size and/or material composition) has occurred and adjust the input signal (and thus the thermal cycle), based on the change in the lithotripsy target.

The input signal can also be adjusted based at least in part on a signal received from a sensor, such as a temperature sensor, a pressure sensor, an imaging or optical sensor, or any similar sensor or device, monitoring a condition at or near the distal end 100 and/or the tip 102 of the elongate member. For example, a temperature sensor can monitor the temperature of the medium in which a distal portion of the elongate member is located. The monitored temperature can be communicated to controller circuitry, which can adjust the input signal to generate or absorb more heat as appropriate or desired based on the monitored temperature. In another example, when multiple thermoelectric transducers or thermocouples are used, at least one of the thermocouples can be used as a temperature sensor. In such an example, the thermocouple, instead of employing a Peltier effect can employ the Seebeck effect. The Seebeck effect can convert temperature difference between two conductors to a voltage difference, thus allowing the thermocouple to be used as a temperature sensor.

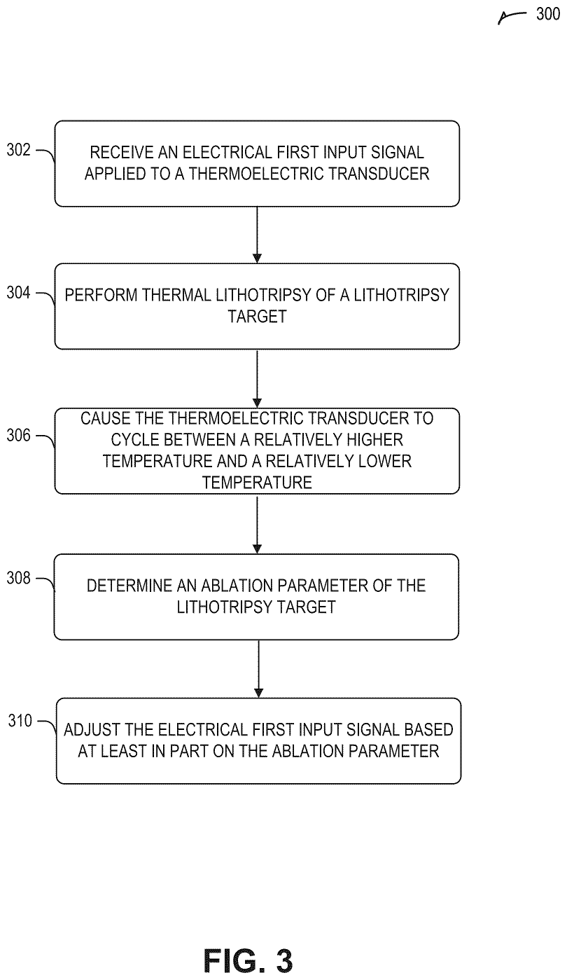

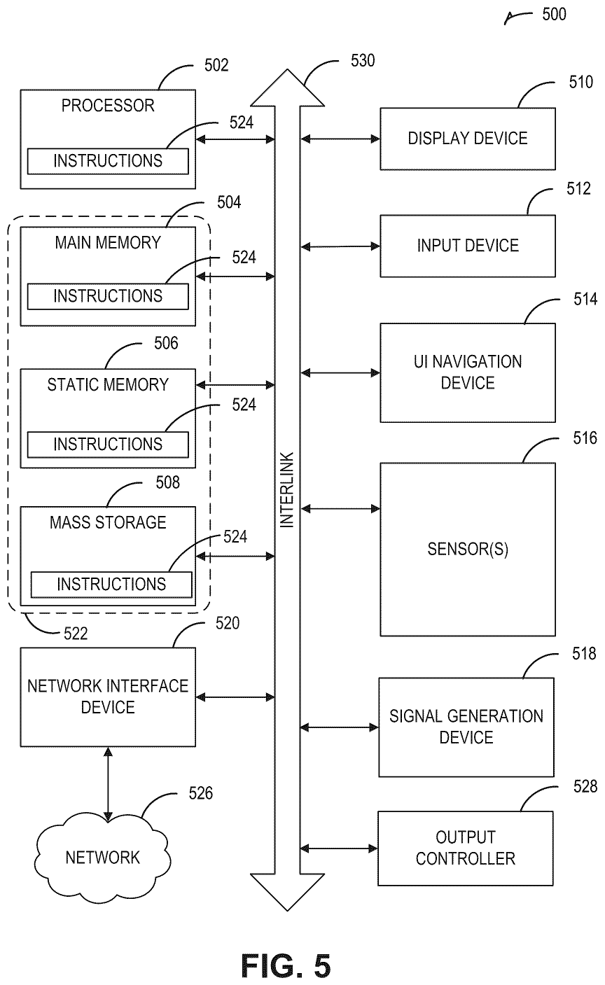

FIG. 3 illustrates an example of a method 300 of ablating a lithotripsy target. The method 300 can include or comprise a number of Operations or Steps (302-310). These Operations are exemplary, and the method can omit one or more of the listed Operations, can repeat Operations, can include other Operations, or can execute the Operations concurrently, substantially simultaneously, or in another order, as appropriate or desired. The operations can be performed automatically by the processor or controller of a machine or computer, such as described below for FIG. 5.

At 302 the method 300 can include receiving an electrical first input signal, (e.g., a square wave), applied to a thermoelectric transducer, such as a thermocouple or any device capable of Peltier heating and cooling. At 304, the method 300 can include performing thermal lithotripsy of a lithotripsy target, such as a kidney stone or other similar calculus. The thermal lithotripsy can include generating or delivering a dose of thermal energy (such as a thermal shockwave) using the thermoelectric transducer, to or at the lithotripsy target.

At 306, the method 300 can include causing the thermoelectric transducer to cycle between a relatively higher temperature and a relatively lower temperature. For example, the thermoelectric transducer can cycle between 0° C. and 50° C., inclusive. The cycle can be controlled using the electrical first input signal, such as by changing an amount of current and/or voltage of the electrical first input signal. The amount of the current and/or voltage of the electrical first input signal can be adjusted using duty cycle or pulse width control of the electrical first input signal to operate the thermoelectric transducer.

At 308, the method 300 can include determining an ablation parameter of the lithotripsy target. The ablation parameter can include a size of the lithotripsy target, a location of the lithotripsy target, a material composition of the lithotripsy target, and/or any other parameter that can affect how much energy should be directed at the lithotripsy target. The ablation parameter can also include an environment around the target such as the type of medium in which the lithotripsy target is located, a temperature and/or pressure of the medium, a location of the lithotripsy target in the anatomy of a patient, or the like. The ablation parameter can be determined using a sensor, such as a temperature or pressure sensor, a composition sensor, an optical sensor, such as a spectrometer, and/or any similar sensor included on, in, or connected to the lithotripsy device.

Depending on the ablation parameters, a tip of a lithotripsy device in which the thermoelectric transducer is included can be located to make physical contact with the target or can be located such that the tip of the lithotripsy device does not contact or touch the target to deliver the dose of thermal lithotripsy. As such, the thermal lithotripsy can be contactless, based on one or more ablation parameters of the target.

At 310, the method 300 can include adjusting the electrical first input signal based at least in part on the ablation parameter. In an example, at 308, a determination may be made that the lithotripsy target is in a location that requires it to be dislodged, and the electrical first input signal can be adjusted such that an intensity of the thermal shockwave discussed above can cause the lithotripsy target to move. In another example, a spectral or other composition analysis of the lithotripsy target may determine that the lithotripsy target is formed from a hard material such as Calcium Oxalate or Calcium Phosphate and adjust the first input signal based on the composition to most efficiently ablate the lithotripsy target.

The lithotripsy target can also change in composition, as the lithotripsy target is ablated. For example, a kidney stone may have a “shell” formed from one type of material and an inner core formed from a different type of material with a harder or softer composition than the shell. In such an example, the amount or intensity of thermal lithotripsy required to reduce or ablate the shell can be different than the amount or intensity of thermal lithotripsy required to ablate the inner core. Thus, the processing circuitry discussed above can adjust (or make a recommendation to a user to adjust) the electrical first input signal to adjust (increase, decrease, or stop) the amount or intensity of thermal lithotripsy being delivered to the target.

Similarly, conditions of the medium and/or the surgical field can change during a medical procedure and the amount or intensity of thermal lithotripsy being delivered to the target can be adjusted based on such changes. For example, the temperature of the medium in which the lithotripsy target is located may rise to an undesired level, and the electrical input signal can be adjusted to cause the thermoelectric transducer to absorb heat and cool the medium, terminate the thermal lithotripsy generation, or the like.

In another example, techniques to affect the surgical field can also be implemented or recommended, such as by the processing circuitry, based on the amount of thermal lithotripsy being delivered to the lithotripsy target. For example, when an amount of thermal lithotripsy energy is being delivered to break up or otherwise ablate the lithotripsy target (as opposed to moving or dislodging the lithotripsy target), irrigation or suction can be activated or adjusted so as to help keep the surgical field clear.

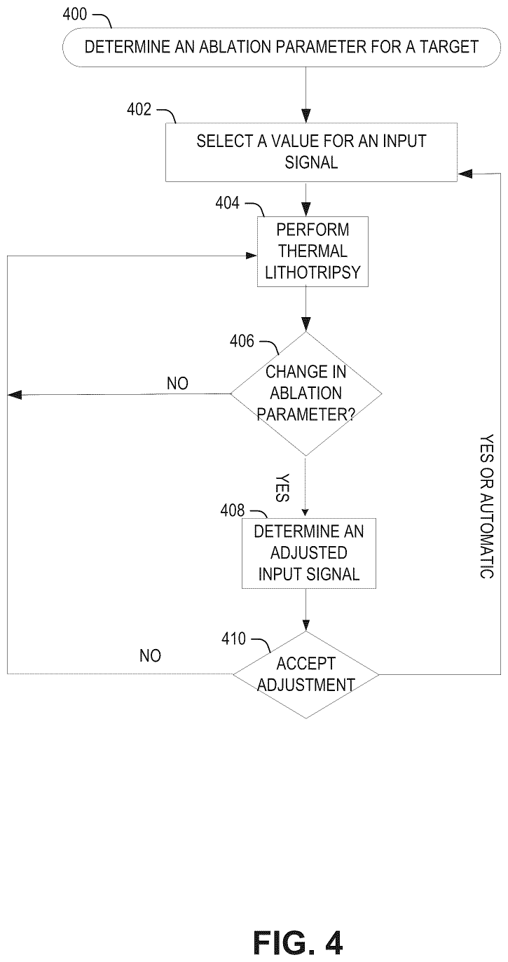

FIG. 4 includes an example flow diagram for ablating a lithotripsy target. At 400, an ablation parameter, such as those described above, can be determined for the lithotripsy target. The ablation parameter can be determined at a first time such as before a therapeutic, ablation, or lithotripsy procedure (e.g., during a diagnostic procedure that occurs before the therapeutic, lithotripsy, or ablation procedure). Alternatively, the ablation parameter can be determined at the beginning of the ablation procedure. For example, the size, location and/or composition of a kidney stone can be determined during a diagnostic procedure or at the beginning of the ablation procedure, prior to ablation of the stone.

At 402, a value for an input signal to be sent to the thermoelectric transducer can be selected or determined, and at 404, thermal lithotripsy (ablation of the stone) can be performed based on the determined ablation parameter and using the selected input signal. For example, based on the size and/or material composition of the stone, an input signal that can allow the thermoelectric transducer to operate at a desired thermal cycle, to provide an appropriate amount of heating or cooling, or the like, can be selected and the ablation procedure started using the selected input signal. The progress of the ablation procedure and/or the status of the target can be monitored throughout the procedure such as through the use of one or more sensors (e.g., a pressure sensor, a temperature sensor, an imaging or optical sensor, or the like). The monitoring can be performed continuously, randomly, periodically, recurrently or at specific time intervals, or at any duration desired or appropriate for the procedure.

The processing circuitry discussed herein utilizing a machine-learning or other algorithm can use data or signals from the monitored sensors to determine at 406 whether, at a second time subsequent to the first time, a change in the ablation parameter has occurred. For example, the processing circuitry can determine whether the stone has been reduced in size, whether the stone has moved to an undesired location, whether the material composition of the stone has changed, or the like, and based on a determined change, at 408, adjust the input signal. In an example, the system can automatically adjust the input signal to change the thermal cycle, increase heat generation or heat absorption of the thermoelectric transducer, cease, stop, or otherwise terminate or cut off the thermal energy being delivered, etc.

Alternatively, the processing circuitry can cause a notification or recommendation to be made, such as on a Graphical User Interface (GUI) to the physician with a recommended changed input signal (or other setting of the electrosurgical device). For example, along with or separate from a recommended changed or updated input signal, the processing circuitry may cause a recommendation to the physician to perform irrigation or suction of the surgical field or adjust the location of the lithotripsy device, or the like. In such an example, at 410 when the recommendation to adjust the input signal is accepted (or not rejected within a period of time), thermal lithotripsy using the current input signal can be resumed or continued (returning to 404). When the recommended adjustment to the input signal is accepted (or when the adjustment is to occur automatically), the new value for the input signal can be selected (returning to 402) and the thermal lithotripsy resumed using the new input signal.

FIG. 5 is a block diagram illustrating an example of a machine upon which one or more embodiments may be implemented. In alternative embodiments, the machine 500 may operate as a standalone device or may be connected (e.g., networked) to other machines. Thus, the machine 500 can include the processing circuitry discussed above and/or be configured to cause the electrical input signal(s) to be provided, transmitted, or sent to the thermoelectric transducer(s), determine the ablation parameter(s) or a change in the ablation parameter(s) of the lithotripsy target, adjust (or provide a recommendation to the user to adjust) the input signal(s), or cause the operations of the processing circuitry discussed herein to be performed.

In a networked deployment, the machine 500 may operate in the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 500 may include a hardware processor 502 (e.g., a central processing unit (CPU), a graphics processing unit (GPU), a hardware processor core, field programmable gate array (FPGA), or any combination thereof), a main memory 504 and a static memory 506, some or all of which may communicate with each other via an interlink (e.g., bus) 530. The machine 500 may further include a display unit 510, an alphanumeric input device 512 (e.g., a keyboard), and a user interface (UI) navigation device 514 (e.g., a mouse). In an example, the display unit 510, input device 512 and UI navigation device 514 may be a touch screen display. The machine 500 may additionally include a storage device (e.g., drive unit) 508, a signal generation device 518 (e.g., a speaker), a network interface device 520, and one or more sensors 516, such as a global positioning system (GPS) sensor, compass, accelerometer, or other sensor. The machine 500 may include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 508 may include a machine readable medium 522 on which is stored one or more sets of data structures or instructions 524 (e.g., software) embodying or used by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the storage device 508 may constitute machine readable media.

While the machine readable medium 522 is illustrated as a single medium, the term “machine readable medium” may include a single medium or multiple media (e.g., a centralized or distributed database, and/or associated caches and servers) configured to store the one or more instructions 524.

The term “machine readable medium” may include any medium that is capable of storing, encoding, or carrying instructions for execution by the machine 500 and that cause the machine 500 to perform any one or more of the techniques of the present disclosure, or that is capable of storing, encoding, or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards), IEEE 802.15.4 family of standards, peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 500, and includes digital or analog communications signals or other intangible medium to facilitate communication of such software.

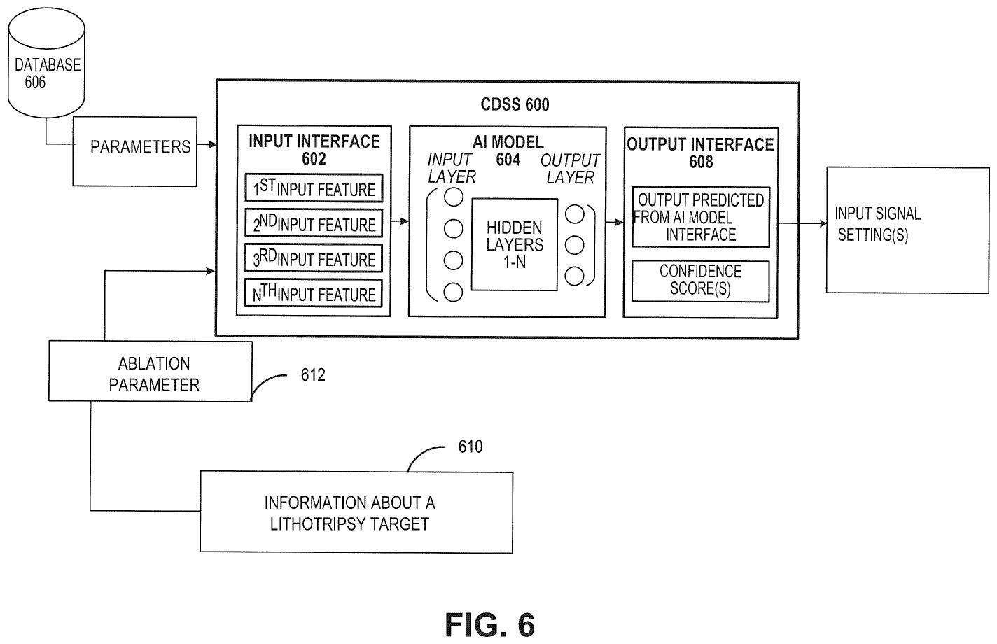

FIG. 6 illustrates a schematic diagram of an exemplary computer-based clinical decision support system (CDSS) 600 that is configured to determine information about (or use information provided about) a target, such as the size of the target, location of the target in the anatomy of a subject or patient, the material composition of the target, or any similar ablation parameters of the target, and select or adjust input signal(s) to be provided to the thermoelectric transducer(s) as described above. In various embodiments, the CDSS 600 includes an input interface 602 through which parameters such as the ablation parameters for the lithotripsy target discussed herein, which are specific to a patient's procedure, are provided as input features to an artificial intelligence (AI) model 604, a processor which performs an inference operation in which the parameters are applied to the AI model to generate the value for the input signal, or an adjusted input signal value to be transmitted or sent to the thermoelectric transducer(s), and an output interface 608 through which the ablation parameter(s), input signal value, and any recommendations can be communicated to a user, e.g., a clinician.

In some embodiments, the input interface 602 may be a direct data link between the CDSS 600 and one or more medical devices that generate at least some of the input features. For example, the input interface 602 may transmit the ablation parameters directly to the CDSS 600 during a therapeutic and/or diagnostic medical procedure. In an example, information about the target and/or the ablation parameters, the devices that are to be used during the therapeutic and/or diagnostic medical procedure can be stored in a database 606. Additionally, or alternatively, the input interface 602 may be a classical user interface that facilitates interaction between a user and the CDSS 600. For example, the input interface 602 may facilitate a user interface through which the user may manually enter the information about the patient, the lithotripsy target, and/or the ablation parameters. Additionally, or alternatively, the input interface 602 may provide the CDSS 600 with access to an electronic patient record from which one or more input features may be extracted. Additionally, or alternatively, the electronic patient record may be stored in the database 606 and transmitted to, retrieved by, or the like, the CDSS 600. In any of these cases, the input interface 602 is configured to collect one or more of the following input features in association with one or more of a specific patient, a type of medical procedure, the devices to be used during the procedure, or the like, on or before a time at which the CDSS 600 is used to assess the information about the target and/or the ablation parameter(s).

An example of an input feature can include information about the lithotripsy device, including the number and type of thermoelectric transducers included in the tip.

An example of an input feature can include data or signals from one or more sensors (e.g., optical sensors, temperature sensors, pressure sensors, or the like) included on or coupled to the lithotripsy device.

An example of an input feature can include information regarding one or more lithotripsy targets at 610.

An example of an input feature can be the type of medium in which the procedure will take place and/or in which the lithotripsy device and/or the target is located.

An example of an input feature can be information regarding an ablation parameter at 612 (or a change in the ablation parameter).

Based on one or more of the above input features, the processor can perform an inference operation using the AI model 604 to select or adjust a value of the input signal(s) to the thermoelectric transducer(s). For example, input interface 602 may deliver the one or more of the input features listed above into an input layer of the AI model 604 which propagates these input features through the AI model 604 to an output layer. The AI model 604 can provide a computer system the ability to perform tasks, without explicitly being programmed, by making inferences based on patterns found in the analysis of data. The AI model 604 explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

Modes for machine learning (ML) can include supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled and allowing the algorithm to act on that information without guidance. Unsupervised ML is useful in exploratory analysis because it can automatically identify structure in data.

Certain tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some tasks for unsupervised ML include clustering, representation learning, and density estimation. Some examples of unsupervised-ML algorithms are K-means clustering, principal component analysis, and autoencoders.

Another type of ML is federated learning (also called collaborative learning) that trains an algorithm across multiple decentralized devices holding local data, without exchanging the data. This approach stands in contrast to other centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model 604 may be trained continuously, recurrently, or periodically prior to performance of the inference operation by the processor. Then, during the inference operation, the patient specific input features provided to the AI model 604 may be propagated from an input layer, through one or more hidden layers, and ultimately to an output layer that corresponds to the information about the target, changes in the target, and the input signal(s) to the transducers to control the amount of thermal lithotripsy energy delivered to the target.

During and/or subsequent to the inference operation, the information about the target and/or the value(s) for the input signals communicated to the user via the output interface 608 (e.g., a user interface (UI)) and/or automatically cause the lithotripsy device to perform a selected action. For example, based on the ablation parameters or changes in the ablation parameters, the system can recommend or automatically select or adjust the input signal to select or change the thermal cycle of the thermoelectric transducer, terminate or otherwise disengage the thermal lithotripsy, cause the scope or tip of the scope to move (or recommend the scope be moved), or the like.

### Additional Notes and Examples

Example 1 is a lithotripsy device for use in a thermal lithotripsy procedure, the lithotripsy device comprising: an elongate member, having a proximal end and a distal end; and a first thermal lithotripsy transducer, at the distal end of the elongate member, to heat and to cool, in response to an applied first electrical input signal, to emit a thermal lithotripsy dose toward a lithotripsy target.

In Example 2, the subject matter of Example 1 optionally includes wherein the first thermal lithotripsy transducer includes a thermoelectric transducer controllable to cycle between first and second target temperatures in response to the applied first electrical input signal, wherein the first and second target temperatures and a frequency of the cycle are specified to promote thermal lithotripsy of the lithotripsy target.

In Example 3, the subject matter of any one or more of Examples 1-2 optionally include subject matter wherein the applied first electrical input signal is a square wave, wherein an amount of a current or a voltage is adjusted using pulse-width modulation control of the electrical first input signal to operate the first thermal lithotripsy transducer for thermal lithotripsy of the lithotripsy target.

In Example 4, the subject matter of any one or more of Examples 1-3 optionally include subject matter wherein the applied first electrical input signal is a square wave, wherein an amount of a current or a voltage is adjusted using duty cycle control of the applied first electrical input signal to operate the first thermal lithotripsy transducer for thermal lithotripsy of the lithotripsy target.

In Example 5, the subject matter of Example 4 optionally includes controller circuitry, operable to at least one of: control a pulsing of the square wave to operate the first thermal lithotripsy transducer for thermal lithotripsy of the lithotripsy target; or control the applied first electrical input signal to cause a thermal cycle to be maintained at a temperature between 0° C. and 50° C. inclusive to operate the first thermal lithotripsy transducer for thermal lithotripsy of the lithotripsy target.

In Example 6, the subject matter of any one or more of Examples 1-5 optionally include subject matter wherein the first thermoelectric transducer includes: a first electrically conductive material; a second electrically conductive material; and a first junction between the first electrically conductive material and the second electrically conductive material, wherein the first thermal lithotripsy transducer is configured for thermal lithotripsy of the lithotripsy target.

In Example 7, the subject matter of Example 6 optionally includes a second thermal lithotripsy transducer at the distal end of the elongate member, wherein the second thermal lithotripsy transducer includes a second thermoelectric transducer configured or configurable to receive an applied second electrical input signal such that the second thermoelectric transducer is operable independent of the first thermoelectric transducer for thermal lithotripsy of the lithotripsy target.

In Example 8, the subject matter of any one or more of Examples 1-7 optionally include subject matter wherein a tip of the distal end of the elongate member has a convex shape and is configured to emit a thermal lithotripsy dose toward a lithotripsy target.

In Example 9, the subject matter of any one or more of Examples 1-8 optionally include subject matter wherein the lithotripsy device is included in or configured to be used with a scope so as to emit the thermal lithotripsy dose toward the lithotripsy target from a distal end of the scope.

In Example 10, the subject matter of Example 9 optionally includes subject matter wherein the scope includes a working channel, and the elongate member is sized and shaped to be inserted via the working channel of the scope so as to emit the thermal lithotripsy dose toward the lithotripsy target from a distal end of the scope.

In Example 11, the subject matter of any one or more of Examples 1-10 optionally include a temperature sensor configured to monitor a temperature at or near the distal end of the elongate member, wherein the temperature sensor is configured to transmit a signal representing the monitored temperature to control circuitry included in or coupled to the lithotripsy device, and wherein the control circuitry is configured to adjust the first electrical input signal, based on the transmitted signal representing the monitored temperature, to control the first thermal lithotripsy transducer to emit the thermal lithotripsy dose toward the lithotripsy target.

Example 12 is a method of thermally ablating a lithotripsy target during a thermal lithotripsy procedure using a scope having a distal end locatable at or near a lithotripsy target, the method comprising: receiving an electrical first input signal applied to a thermal lithotripsy transducer locatable at the distal end of the scope; performing thermal lithotripsy of the lithotripsy target, including by causing the thermal lithotripsy transducer to actively heat and to actively cool to produce and emit a thermal lithotripsy dose toward the lithotripsy target.

In Example 13, the subject matter of Example 12 optionally includes controlling the electrical first input signal to cause the thermal lithotripsy transducer to cycle between a relatively higher temperature and a relatively lower temperature for performing thermal lithotripsy of the lithotripsy target.

In Example 14, the subject matter of Example 13 optionally includes controlling the electrical first input signal to cause the thermal lithotripsy transducer to cycle between a relatively higher temperature and a relatively lower temperature that is between 0° C. and 50° C., inclusive, for performing thermal lithotripsy of the lithotripsy target.

In Example 15, the subject matter of any one or more of Examples 13-14 optionally include subject matter wherein an amount of a current or a voltage of the electrical first input signal is adjusted using duty cycle control of the electrical first input signal to operate the thermal lithotripsy transducer for performing thermal lithotripsy of the lithotripsy target, wherein the applied first input electrical signal is a square wave.

In Example 16, the subject matter of any one or more of Examples 12-15 optionally include determining a thermal lithotripsy ablation parameter of the lithotripsy target; and adjusting a frequency or a period of the electrical first input signal to control a thermal cycle, based at least in part on the thermal lithotripsy ablation parameter for performing thermal lithotripsy of the lithotripsy target.

Example 17 is a lithotripsy device for use in a thermal lithotripsy procedure, the lithotripsy device comprising: a first thermal lithotripsy transducer locatable at a distal end of an elongate member, wherein the first thermal lithotripsy transducer is configurable to heat and to cool, in response to an applied first electrical input signal, to emit a thermal lithotripsy dose toward a lithotripsy target; and controller circuitry operable to at least one of: control the applied first electrical input signal to cause a first thermal cycle of the thermal lithotripsy transducer at a temperature between 0° C. and 50° C. inclusive; or adjust the applied first electrical input signal based on at least one of a signal received from a sensor or an ablation parameter of the lithotripsy target.

In Example 18, the subject matter of Example 17 optionally includes a second thermal lithotripsy transducer at the distal end of the elongate member, wherein the second thermal lithotripsy transducer is configurable to receive an applied second electrical input signal such that the second thermal lithotripsy transducer is operable independent of the first thermal lithotripsy transducer for thermal lithotripsy of the lithotripsy target, wherein the controller circuitry is further operable to: control a pulse width or a duty cycle of at least one of the applied first electrical input signal or the applied second electrical input signal.

In Example 19, the subject matter of Example 18 optionally includes subject matter wherein each of the first thermal lithotripsy transducer and the second thermal lithotripsy transducer includes: a first electrically conductive material; a second electrically conductive material; and a first junction between the first electrically conductive material and the second electrically conductive material, wherein the first thermal lithotripsy transducer is configured for thermal lithotripsy of the lithotripsy target.

In Example 20, the subject matter of any one or more of Examples 17-19 optionally include subject matter wherein the controller circuitry is operable to adjust the applied first electrical input signal, based on the ablation parameter of the lithotripsy target, wherein the ablation parameter includes at least one of a size of the lithotripsy target, a material composition of the lithotripsy target, a change in size of the lithotripsy target, or a change in material composition of the lithotripsy target, and wherein the ablation parameter is determined by one or more of: a signal received from a temperature sensor, a signal received from a composition sensor, or spectral analysis of a portion of emitted light returned from or reflected off the lithotripsy target.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A lithotripsy device for use in a thermal lithotripsy procedure, the lithotripsy device comprising:
an elongate member, having a proximal end and a distal end; and
a first thermal lithotripsy transducer, at the distal end of the elongate member, to heat and to cool, in response to an applied first electrical input signal, to emit a thermal lithotripsy dose toward a lithotripsy target.

2. The lithotripsy device of claim 1, wherein the first thermal lithotripsy transducer includes a thermoelectric transducer controllable to cycle between first and second target temperatures in response to the applied first electrical input signal, wherein the first and second target temperatures and a frequency of the cycle are specified to promote thermal lithotripsy of the lithotripsy target.

3. The lithotripsy device of claim 1, wherein the applied first electrical input signal is a square wave, wherein an amount of a current or a voltage is adjusted using pulse-width modulation control of the applied first electrical input signal to operate the first thermal lithotripsy transducer for thermal lithotripsy of the lithotripsy target.

4. The lithotripsy device of claim 1, wherein the applied first electrical input signal is a square wave, wherein an amount of a current or a voltage is adjusted using duty cycle control of the applied first electrical input signal to operate the first thermal lithotripsy transducer for thermal lithotripsy of the lithotripsy target.

5. The lithotripsy device of claim 4, further comprising:
controller circuitry, operable to at least one of:
control a pulsing of the square wave to operate the first thermal lithotripsy transducer for thermal lithotripsy of the lithotripsy target; or
control the applied first electrical input signal to cause a thermal cycle to be maintained at a temperature between 0° C. and 50° C. inclusive to operate the first thermal lithotripsy transducer for thermal lithotripsy of the lithotripsy target.

6. The lithotripsy device of claim 1, wherein the first thermal lithotripsy transducer includes:
a first electrically conductive material;
a second electrically conductive material; and
a first junction between the first electrically conductive material and the second electrically conductive material, wherein the first thermal lithotripsy transducer is configured for thermal lithotripsy of the lithotripsy target.

7. The lithotripsy device of claim 6, comprising:
a second thermal lithotripsy transducer at the distal end of the elongate member, wherein the second thermal lithotripsy transducer includes a second thermoelectric transducer configured or configurable to receive an applied second electrical input signal such that the second thermal lithotripsy transducer is operable independent of the first thermal lithotripsy transducer for thermal lithotripsy of the lithotripsy target.

8. The lithotripsy device of claim 1, wherein a tip of the distal end of the elongate member has a convex shape and is configured to emit a thermal lithotripsy dose toward a lithotripsy target.

9. The lithotripsy device of claim 1, wherein the lithotripsy device is included in or configured to be used with a scope so as to emit the thermal lithotripsy dose toward the lithotripsy target from a distal end of the scope.

10. The lithotripsy device of claim 9, wherein the scope includes a working channel, and the elongate member is sized and shaped to be inserted via the working channel of the scope so as to emit the thermal lithotripsy dose toward the lithotripsy target from a distal end of the scope.

11. The lithotripsy device of claim 1, comprising:
a temperature sensor configured to monitor a temperature at or near the distal end of the elongate member, wherein the temperature sensor is configured to transmit a signal representing the monitored temperature to control circuitry included in or coupled to the lithotripsy device, and wherein the control circuitry is configured to adjust the applied first electrical input signal, based on the transmitted signal representing the monitored temperature, to control the first thermal lithotripsy transducer to emit the thermal lithotripsy dose toward the lithotripsy target.

12. A method of thermally ablating a lithotripsy target during a thermal lithotripsy procedure using a scope having a distal end locatable at or near a lithotripsy target, the method comprising:
receiving an electrical first input signal applied to a thermal lithotripsy transducer locatable at the distal end of the scope; and
performing thermal lithotripsy of the lithotripsy target, including by causing the thermal lithotripsy transducer to actively heat and to actively cool to produce and emit a thermal lithotripsy dose toward the lithotripsy target.

13. The method of claim 12, comprising controlling the electrical first input signal to cause the thermal lithotripsy transducer to cycle between a relatively higher temperature and a relatively lower temperature for performing thermal lithotripsy of the lithotripsy target.

14. The method of claim 13, comprising controlling the electrical first input signal to cause the thermal lithotripsy transducer to cycle between a relatively higher temperature and a relatively lower temperature that is between 0° C. and 50° C., inclusive, for performing thermal lithotripsy of the lithotripsy target.

15. The method of claim 13, wherein an amount of a current or a voltage of the electrical first input signal is adjusted using duty cycle control of the electrical first input signal to operate the thermal lithotripsy transducer for performing thermal lithotripsy of the lithotripsy target, wherein the electrical first input signal is a square wave.

16. The method of claim 12, comprising:
determining a thermal lithotripsy ablation parameter of the lithotripsy target; and
adjusting a frequency or a period of the electrical first input signal to control a thermal cycle, based at least in part on the thermal lithotripsy ablation parameter for performing thermal lithotripsy of the lithotripsy target.

17. A lithotripsy device for use in a thermal lithotripsy procedure, the lithotripsy device comprising:
a first thermal lithotripsy transducer locatable at a distal end of an elongate member, wherein the first thermal lithotripsy transducer is configurable to heat and to cool, in response to an applied first electrical input signal, to emit a thermal lithotripsy dose toward a lithotripsy target; and
controller circuitry operable to at least one of:
control the applied first electrical input signal to cause a first thermal cycle of the first thermal lithotripsy transducer at a temperature between 0° C. and 50° C. inclusive; or
adjust the applied first electrical input signal based on at least one of a signal received from a sensor or an ablation parameter of the lithotripsy target.

18. The lithotripsy device of claim 17, further comprising:
a second thermal lithotripsy transducer at the distal end of the elongate member, wherein the second thermal lithotripsy transducer is configurable to receive an applied second electrical input signal such that the second thermal lithotripsy transducer is operable independent of the first thermal lithotripsy transducer for thermal lithotripsy of the lithotripsy target, wherein the controller circuitry is further operable to:
control a pulse width or a duty cycle of at least one of the applied first electrical input signal or the applied second electrical input signal.

19. The lithotripsy device of claim 18, wherein each of the first thermal lithotripsy transducer and the second thermal lithotripsy transducer includes:
a first electrically conductive material;
a second electrically conductive material; and
a first junction between the first electrically conductive material and the second electrically conductive material, wherein the first thermal lithotripsy transducer is configured for thermal lithotripsy of the lithotripsy target.

20. The lithotripsy device of claim 17, wherein the controller circuitry is operable to adjust the applied first electrical input signal, based on the ablation parameter of the lithotripsy target, wherein the ablation parameter includes at least one of a size of the lithotripsy target, a material composition of the lithotripsy target, a change in size of the lithotripsy target, or a change in material composition of the lithotripsy target, and wherein the ablation parameter is determined by one or more of: a signal received from a temperature sensor, a signal received from a composition sensor, or spectral analysis of a portion of emitted light returned from or reflected off the lithotripsy target.
